(19) United States
(12) Patent Application Publication
(10) Pub. No.: US 2009/0042790 A1
(43) Pub. Date: Feb. 12, 2009

(54) TRANSMUCOSAL DELIVERY OF PEPTIDE DERIVATIVES

(75) Inventors: Henry R. Costantino, Woodinville, WA (US); Mary S. Kleppe, Oaksdale, CT (US); Ching-Yuan Li, Bellevue, WA (US)

Correspondence Address: NASTECH PHARMACEUTICAL COMPANY INC;MDRNA, Inc., 3830 MONTE VILLA PARKWAY, BOTHELL, WA, 98021-7266, US

(73) Assignee: NASTECH PHARMACEUTICAL COMPANY INC., Bothell, WA (US)

(21) Appl. No.: 11/917,340

(22) PCT Filed: Jun. 13, 2006
(86) PCT No.: PCT/US06/23220

§ 371 Date: Aug. 28, 2008

Related U.S. Application Data
(60) Provisional application No. 60/689,869, filed on Jun. 13, 2005.
(60) Provisional application No. 60/690,842, filed on Jun. 14, 2005.
(60) Provisional application No. 60/748,946, filed on Dec. 9, 2005.

(51) Int. Cl. A61K 38/00; C07K 14/00; A61P 3/00

(52) U.S. Cl. 514/12; 530/324

(57) ABSTRACT

What is described is a biological agent, comprised of a biologically active protein, or a fragment or a mimetic thereof, conjugated to at least one poly(alkylene oxide) chain having a size less than about 20 kDa, pharmaceutical formulations for intranasal delivery of said biological agent, or uses of said biological agent in the manufacture of said pharmaceutical formulation for administering said biological agent to a mammal.

### BACKGROUND OF THE INVENTION

Obesity is a growing epidemic among adults and children in the United States and other parts of the world. It is associated with significant health risks including cardiovascular disease, diabetes, and orthopedic problems and consequently places an unneeded burden upon the health care system. Recent evidence suggests a link between obesity in humans and mutations in the melanocortin-4 (MC4) receptor (Yeo et. al., Nat. Genet., 20(2): 111-112, 1998; Vaisse et. al., Nat. Genet., 20(2):113-114, 1998). MC4 receptor is bound and activated by the peptide hormone melanocortin. Animal studies show a direct role for MC4 receptor in body weight regulation. Targeted disruption of the MC4 receptor locus in mice results in obesity while administration of a melanocortin agonist to wild-type mice inhibited food intake and increased energy expenditure. Taken together, these data indicate that MC4 receptor is an ideal pharmacological target for treating obesity in humans. Suitable, potential therapeutic candidates in this regard are MC4 and fragments of MC4 that retain the ability to bind and activate the MC4 receptor, and also MC4 receptor agonists (MC4-RA), that is, peptides or other low-molecular-weight compounds that exhibit the ability to bind and activate the MC4 receptor.

In addition to the requisite biological activity, it is also necessary that the dosage form of MC-4, MC-4 analog, MC-4 fragment, or MC-4RA is suitable for delivery to the obese subject. Oral administration would not provide a suitable option for peptides and proteins, which exhibit very low bioavailability when given by this route due to hepatic first-pass metabolism and degradation in the gastrointestinal tract. A major disadvantage of drug administration by injection is that trained personnel are often required to administer the drug. For self-administered drugs, many patients are reluctant or unable to give themselves injections on a regular basis. Injection is also associated with increased risks of infection. Other disadvantages of drug injection include variability of delivery results between individuals, as well as unpredictable intensity and duration of drug action.

Conjugation with water-soluble polymers such as poly(ethylene glycol) (PEG) and derivatives of PEG have been used as a strategy to enhance the half life of protein pharmaceuticals, in particular for injected dosage forms (Caliceti P., et al., Adv Drug Deliv. Rev., 55:1261-77, 2003). Other potential benefits of modification of peptides and proteins with polymers such as PEG include chemical (Diwan M., et al., Int J Pharm., 252:111-22, 2003) and biochemical stabilization (Na D. H., et al., J Pharm Sci., 93:256-261, 2004) and attenuation of immunogenicity (Yang Z., et al., Cancer Res., 64:6673-8, 2004). However, relatively few reports have explored utilization of PEGylated peptides and proteins non non-injection delivery. For example, U.S. Pat. No. 6,565,841 describes delivery of PEGylated pulmonary delivery of granulocyte colony stimulating factor. A further example is U.S. Pat. No. 6,165,509 describes PEGylated drugs complexed with bioadhesive polymers for delivery to mucosal surfaces. In another example, mono-PEGylation to the peptide salmon calcitonin results in increased intranasal bioavailability in rats, with the enhancement being inversely proportional to the PEG molecular weight (MW) (Lee K. C., et al., Calcif Tissue Int., 73:545-9, 2003; Shin B. S., et al., Chem Pharm Bull (Tokyo), 52:957-60, 2004), hereby incorporated by reference in their entirety. These publications represent a limited approach to enhancing bioavailability of calcitonin, namely by increasing the lifetime of molecules in the biological organism. There is an unmet need to increase bioavailability of biologically active molecules by other means.

Mucosal administration of therapeutic compounds may offer certain advantages over injection and other modes of administration including convenience and speed of delivery, as well as by reducing or elimination compliance problems and side effects that attend delivery by injection. However, intranasal mucosal delivery of biologically active agents and other therapeutic compounds, including large molecule drugs, peptides and proteins is limited by mucosal barrier functions.

The ability of drugs to permeate mucosal surfaces, unassisted by delivery-enhancing agents, appears to be related to a number of factors, including molecular size, lipid solubility, and ionization. Small molecules, less than about 300-1,000 Daltons, are often capable of penetrating mucosal barriers, however, as molecular size increases, permeability decreases rapidly. Lipid-soluble compounds are generally more permeable through mucosal surfaces than are non-lipid-soluble molecules. Peptides and proteins are poorly lipid soluble, and hence exhibit poor absorption characteristics across mucosal surfaces.

Previous attempts to successfully deliver therapeutic compounds, including small molecule drugs and protein therapeutics, via mucosal routes have suffered from a number of important and confounding deficiencies. These deficiencies point to a long-standing unmet need in the art for pharmaceutical formulations and methods of administering therapeutic compounds that are stable and well tolerated and that provide enhanced mucosal delivery, including to targeted tissues and physiological compartments such as central nervous system. More specifically, there is a need in the art for safe and reliable methods and compositions for mucosal delivery of therapeutic compounds for treatment of diseases and other adverse conditions in mammalian subjects. A related need exists for methods and compositions that will provide efficient delivery of macromolecular drugs via one or more mucosal routes in therapeutic amounts, which are fast acting, easily administered and have limited adverse side effects such as mucosal irritation or tissue damage.

Selective permeability of mucosal epithelia has heretofore presented major obstacles to mucosal delivery of therapeutic macromolecules, including biologically active peptides and proteins. Accordingly, there is a compelling need in the art for new methods and formulations to facilitate mucosal delivery of biotherapeutic compounds that have heretofore proven refractory to delivery across mucosal barriers. Through pharmaceutical formulations that incorporate specific permeation enhancers with a therapeutic agent, the present invention satisfies these needs.

### BRIEF DESCRIPTION OF THE DRAWINGS

FIG. 1: The ability of the 2 kDa PEGylated MC4-RA and unmodified MC-4RA to stimulate cAMP production in cells expressing the melanocortin-4 cell receptor (MC4 receptor) was compared. HEK293 cells expressing the MC4 receptor were incubated with either the 2 kDa PEGylated MC4-RA or the unmodified MC4-RA. The concentrations used for either MC4-RA in the cAMP assay ranged from approximately 1×10⁻¹¹ to 1×10⁻⁵ M. The maximum quantity of cAMP was normalized to 100% or “% Max Response” and the concentration of 2 kDA PEGylated or unmodified MC4-RA was shown as the log of the Molar concentration. The effective concentration to achieve a 50% response (EC₅₀) is shown. The 2 kDA PEGylated MC4-RA is a less potent activator of the MC-4 receptor compared to the unmodified MC4-RA in an in vitro assay system.

FIG. 2: The degree of MC4 receptor specificity of the 2 kDa PEGylated MC4-RA and the unmodified MC4-RA was compared. The ability to stimulate cAMP production in cells in vitro was assayed; HEK293 cells expressed the melanocortin-1 (MC1 receptor). A measured increase in cAMP levels indicated a lack of MC4 receptor specificity. The 2 kDa PEGylated and unmodified MC4-RA were incubated in a concentration range of approximately 1×10⁻¹¹ to 1×10⁻⁵ M with HEK293 cells that were expressing the MC1 receptor. The maximum quantity of cAMP was normalized to 100% or “% Max Response” and the concentration of 2 kDA PEGylated or unmodified MC4-RA was shown as the log of the Molar concentration. The effective concentration to achieve a 50% response (EC₅₀) is shown. PEGylation significantly enhanced the specificity of MC4-RA for the MC4 receptor.

FIG. 3: The effect of the unmodified MC4-RA on food intake was evaluated 16 and 24 hours after dose administration. The high dose group for the unmodified MC4-RA showed significant reduction on cumulative food intake 16 and 24 hours after dose administration.

FIG. 4: The effect of the low molecular weight PEGylated MC4-RA on food intake was evaluated 16 and 24 hours after dose administration. The high dose group for the 2 kDA PEGylated MC4-RA showed significant reduction on cumulative food intake 16 and 24 hours after dose administration.

### DESCRIPTION OF THE INVENTION

The current invention relates to the use of a MC4-RA conjugated to a low molecular weight PEG moiety for enhanced mucosal delivery in the treatment of disease. In vitro assessment indicates that low molecular weight PEG conjugation to a MC4-RA enhances permeation of the agonist across an epithelial cell monolayer. Further, in vivo administration of a low molecular weight PEG conjugated to a MC4-RA significantly reduced cumulative food intake in mammalian subjects. Thus, conjugation of a low molecular weight PEG to a MC4-RA represents a promising new therapeutic approach for improving the delivery of a MC4-RA for the treatment of a wide range of diseases and disorders, for example obesity.

The invention includes formulations for enhancing cellular permeability of a molecule, comprising a molecule and an enhancer of cellular permeation, wherein such molecule is conjugated to at least one water soluble polymer. The preferred water-soluble polymer is selected from the group consisting of poly(alkylene oxide). Preferred poly(alkylene oxides) are selected from the group consisting of alpha-substituted poly(alkylene oxide) derivatives, PEG homopolymers and derivatives thereof, poly(propylene glycol) (PPG) homopolymers and derivatives thereof, poly(ethylene oxides) (PEO) polymers and derivatives thereof, bis-poly(ethylene oxides) and derivatives thereof, copolymers of poly(alkylene oxides), and block copolymers of poly(alkylene oxides), poly(lactide-co-glycolide) and derivatives thereof, or activated derivatives thereof. Preferably, the water-soluble polymer has a molecular weight of about 200 to about 40,000 Da, more preferably about 200 to about 10,000 Da, most preferably about 200 to 5,000 Da. The preferred water-soluble polymers are poly(alkylene oxides), most preferably PEG or poly(ethylene oxide) (PEO).

Preferably, the molecule (of therapeutic use to a mammal) is a peptide or protein consisting of 2-500 amino acid residues, more preferably 2-100 amino acid residues, most preferably 2-50 amino acid residues. Preferably, the peptide or protein may be monomeric or oligomeric, for example dimeric. The peptide or protein monomers may form the dimers or higher-order oligomers by physical or chemical means. The conjugate may be resistant to physiological processes, including proteolysis, enzyme action or hydrolysis in general. Alternatively, the conjugate can be cleaved by processes of biodegradation, for example a pro-drug approach. Preferably, the molecule is covalently linked to a single poly(alkylene oxide) chain, which may be unbranched or branched, most preferably, unbranched. The means of conjugation are generally known to ordinary skilled workers (see U.S. Pat. No. 5,595,732; U.S. Pat. No. 5,766,897; U.S. Pat. No. 5,985,265; U.S. Pat. No. 6,528,485; U.S. Pat. No. 6,586,398; U.S. Pat. No. 6,869,932; and U.S. Pat. No. 6,706,289, hereby incorporated by reference in their entirety).

One aspect of the invention is a formulation for enhancing cellular permeability of a molecule, comprising a molecule and an enhancer of cellular permeation, wherein such molecule is conjugated to at least one poly(allylene oxide) chain. A related embodiment is a formulation, wherein the molecule is covalently linked to a single poly(alkylene oxide) chain.

An embodiment of the invention is a formulation for enhancing cellular permeability of a molecule, wherein the poly(alkylene oxide) chain is a PEG chain. Covalent attachment PEG to a polypeptide is disclosed in U.S. Pat. No. 4,179,337 to Davis et al., as well as in Abuchowski and Davis “Enzymes as Drugs,” Holcenberg and Roberts, Eds., pp. 367-383, John Wiley and Sons, New York (1981), hereby incorporated by reference in their entirety. A related embodiment is a PEG that has a molecular size between about 0.2 and about 200 kiloDaltons (kDa). A related embodiment is a PEG that has a molecular size less than 40 kDa, preferably less than 20 kDa, more preferably less than 10 kDa, more preferably less than 5 kDa, and most preferably, less than 2 kDa.

Another embodiment of the invention is a formulation for enhancing cellular permeability of a molecule by decreasing electrical resistance across a cellular layer. The cellular layer can be an endothelial cell layer or an epithelial cell layer. Epithelial cells include mucosal cells, such as nasal, bronchial, bucal, or gastrointestinal cells. The enhancer of permeation increases permeability of the molecule across a cellular layer, preferably a monocellular layer. Increased permeation may be paracellular, for example through tight junctions, and between cells. Alternatively, permeation is enhanced through the cell, for example, through endocytosis or pinocytosis.

The enhancer of cellular permeation may include molecules that are known to modify tight junctions, e.g., chelating agents, such as EDTA, or specific tight junction modifiers (TJM) as PN159 or other known TJM (see Johnson and Quay (2005) Expert Opinion Drug Delivery 2:281-98, hereby incorporated by reference in its entirety).

The enhancer of cellular permeability may comprise a solubilizing agent, for example, cyclodextran, hydroxypropyl-β-cyclodextran, sulfobutylether-β-cyclodextran and methyl-β-cyclodextrin, most preferably methyl-β-cyclodextrin.

The enhancer of cellular permeability may include a surface active agent, for example a nonionic polyoxyethylene ether, bile salts such as sodium glycocholate (SGC), deoxycholate (DOC), derivatives of fusidic acid, or sodium taurodihydrofusidate (STDHF), L-α-phosphatidylcholine dodecanoyl (DDPC), polysorbate 80 and polysorbate 20,), cetyl alcohol, polyvinylpyrolidone (PVP), polyvinyl alcohol (PVA), lanolin alcohol, and sorbitan monooleate. Most preferably, the surface active agent is DDPC.

The enhancer of cellular permeability may include one or more polyols, most preferably at least two polyols. The polyols are selected preferably selected from the group consisting of sucrose, mannitol, sorbitol, lactose, trehalose, L-arabinose, D-erythrose, D-ribose, D-xylose, D-mannose, trehalose, D-galactose, lactulose, cellobiose, gentiobiose, glycerin and polyethylene glycol, and most preferably, lactose and sorbitol.

Another embodiment of the invention is a formulation for enhancing cellular permeability of a molecule, wherein the formulation has a pH from about 3.0 to about pH 8.0, preferably a pH from 3.0 to 6.0, and most preferably a pH from 3.0 to 5.0.

Another embodiment of the invention is a method of administering a molecule to an animal comprising preparing a formulation, described supra, and bringing such formulation in contact with a mucosal surface of such animal. These include, for example, bucal, gastrointestinal, nasal, epidermal, and bronchial surfaces. Most preferably, administration is by contact with an intranasal surface.

The dosage form may be liquid or solid. If liquid it may be administered to the mucosal surface as a spray, said spray generated by techniques know to the art such as atomization and nebulization, or the liquid may be instilled into the mucosal surface. If a solid or semi-solid, it may be reconstituted to a liquid by addition of water, and then administered to the mucosal surface as described above, or the solid or semi-solid may be applied directly to the mucosal surface. Techniques know in the art such as freeze drying, spray drying, spray-freeze drying, supercritical fluid drying, rotary and film evaporation and the like may be used to produce the dried material. The solid or semi-solid formulation may alternatively be present in a capsule or tablet.

### EXAMPLES

The above disclosure generally describes the present invention, which is further exemplified by the following examples. These examples are described solely for purposes of illustration, and are not intended to limit the scope of the invention. Although specific terms and values have been employed herein, such terms and values will likewise be understood as exemplary and non-limiting to the scope of the invention.

### Example 1

### Protocols and Methods

The present example illustrates the reagents, methods, protocols and the source of each used in the subsequent Examples of the instant application.

### Cell Cultures

The EpiAirway™ system was developed by MatTek Corp. (Ashland, Mass.) as a model of the pseudostratified epithelium lining the respiratory tract. The epithelial cells are grown on porous membrane-bottomed cell culture inserts at an air-liquid interface, which results in differentiation of the cells to a highly polarized morphology. The apical surface is ciliated with a microvillous ultrastructure and the epithelium produces mucus (the presence of mucin has been confirmed by immunoblotting). The inserts have a diameter of 0.875 cm, providing a surface area of 0.6 cm². The cells are plated onto the inserts at the factory approximately three weeks before shipping. One “kit” consists of 24 units.

EpiAirway™ culture membranes were received the day before the experiments started. They are shipped in phenol red-free and hydrocortisone-free Dulbecco's Modified Eagle's Medium (DMEM). The cells were provided as inserts grown to confluent on Millipore Millicell-CM filters comprised of transparent hydrophilic Teflon (PTFE). Each tissue insert was placed into a well of a 6 well plate containing 1 ml of serum free DMEM. The membranes were then cultured for 24 hrs at 37° C./5% CO₂ to allow tissues to equilibrate. This DMEM-based medium is serum free but is supplemented with epidermal growth factor and other factors. The medium is always tested for endogenous levels of any cytokine or growth factor which is being considered for intranasal delivery, but has been free of all cytokines and factors studied to date except insulin. The volume is sufficient to provide contact to the bottoms of the units on their stands, but the apical surface of the epithelium is allowed to remain in direct contact with air. Sterile tweezers are used in this step and in all subsequent steps involving transfer of units to liquid-containing wells to ensure that no air is trapped between the bottoms of the units and the medium.

This model system was used to evaluate the effect of a low molecular weight poly(ethylene glycol) PEG-MC4-RA conjugate on TEER and permeation. These assays are described below in detail.

### Tissue Permeation Assay

The quantity of MC4-RA and PEGylated MC4-RA conjugate that passed from the apical surface to the basolateral surface of the EpiAirway™ epithelial cell monolayer represented the degree of permeation. Each tissue insert was placed in an individual well containing 0.25 ml of basal media. On the apical surface of the inserts, 50 ml of test formulation containing either MC4-RA or MC4-RA conjugated with PEG was applied, and the samples were placed on a shaker. (˜100 rpm) for 120 minutes at 37° C. A 200 μl sample was taken from the apical and basal side of each insert and placed into a 1.5 ml tube. Tubes were then spun down, at 2,500 rpm for 5 minutes and immediately used for analysis or placed in −20° C. freezer. To prepare the inserts for post TEER reading, an additional 100 μl of fresh media was added to the apical side of each insert and TEER measured and recorded.

Transepithelial electrical resistance (TEER) was measured before and after the two hour incubation.

### Transepithelial Electrical Resistance (TEER):

Respiratory airway epithelial cells form tight junctions in vivo as well as in vitro, and thereby restrict the flow of solutes across the tissue. These junctions confer a transepithelial resistance of several hundred ohms×cm² in excised airway tissues.

Accurate determinations of TEER require that the electrodes of the ohmmeter be positioned over a significant surface area above and below the membrane, and that the distance of the electrodes from the membrane be reproducibly controlled. The method for TEER determination recommended by MatTek and employed for all experiments herein employs an “EVOM”™ epithelial voltammeter and an “ENDOHM” ™ tissue resistance measurement chamber from World Precision Instruments, Inc., Sarasota, Fla.

The electrodes and a tissue culture blank insert will be equilibrated for at least 20 minutes in fresh media with the power off prior to checking calibration. The background resistance will be measured with 1.5 ml media in the Endohm tissue chamber and 300 μl media in a blank Millicell-CM insert. The top electrode is adjusted so that is submerged in the media but not making contact with the top surface of the insert membrane. Background resistance of the blank insert should be 5 to 20 ohms. For each TEER determination, 300 μl media will be added to the insert followed by 20 minutes incubation at room temperature before placement in the Endohm chamber to read TEER. Measurements were recorded at time zero and then again one hour after exposure to formulations. Resistance was expressed as (resistance measured−blank)×0.6 cm². All TEER values are reported as a function of the surface area of the tissue.

TEER was calculated as:

TEER=(R_{I}−R_{b})×A

Where R_{I} is resistance of the insert with a membrane, R_{b} is the resistance of the blank insert, and A is the area of the membrane (0.6 cm²). A decrease in TEER value relative to the control value (control=approximately 1000 ohms-cm²; normalized to 100.) indicates a decrease in cell membrane resistance and an increase in mucosal epithelial cell permeability.

### Chemical Structure of an Exemplary Cyclic Melanocortin-4 Receptor Agonist (MC4-RA) of the Present Invention

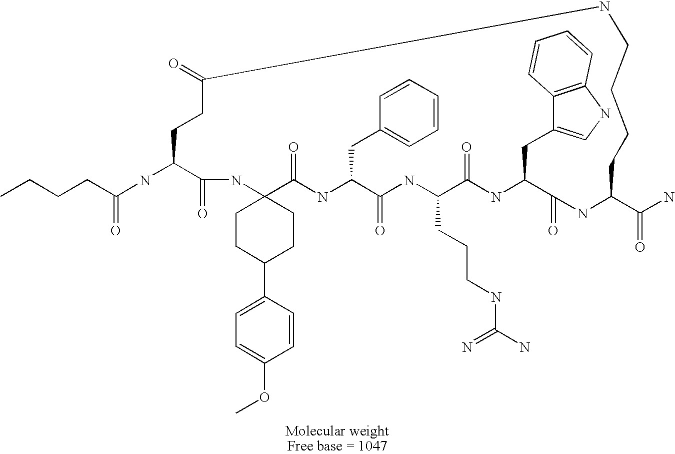

### Chemical Structure of an Exemplary Low Molecular Weight PEGylated Melanocortin-4 Receptor Agonist of the Present Invention (2 kDa PEG-MC4-RA)

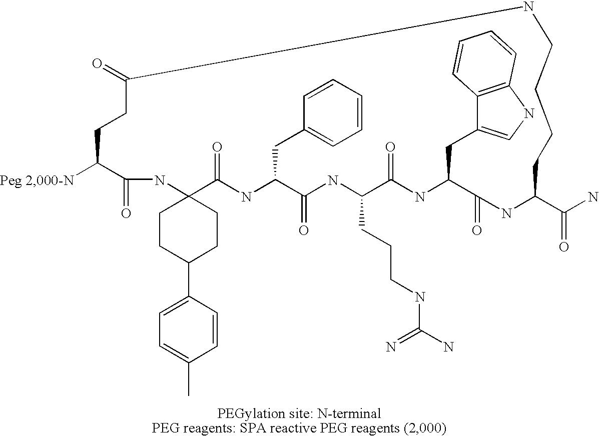

### Example 2

### Permeation Kinetics of PEGylated and Unmodified Forms of an EPO-Mimetic Peptide in the Presence or Absence of Low Molecular Weight Excipients

The present example demonstrates that conjugation of an EPO-mimetic peptide with PEG enhances the permeation of EPO-mimetic peptide across an epithelial cell monolayer. The instant example compares the permeation kinetics of a 5 kDa PEGylated EPO-mimetic peptide with an unmodified EPO-mimetic peptide in the presence or absence of low molecular weight excipients. The results from two separate sets of low molecular weight excipient containing formulations with either 5 kDa PEGylated EPO-mimetic peptide or unmodified EPO-mimetic peptide are shown. Table 1 below illustrates one set of PEGylated and unmodified EPO-mimetic peptide formulations assayed for TEER and epithelial cell monolayer permeation and Table 3 below shows the second set of PEGylated and unmodified EPO-mimetic peptide formulations assayed for TEER and epithelial cell monolayer permeation. Results for formulations shown in Table 1 are summarized in Table 2 and results for formulations shown in Table 3 are summarized in Table 4.

Table 1 below illustrates PEGylated and unmodified EPO-mimetic peptide formulations. These formulations contained 120 μM of 5 kDa PEGylated or 120 μM unmodified EPO-mimetic peptide with or without the low molecular weight excipients methyl-β-cyclodextrin (M-β-CD), disodium edentate (EDTA) and 1-α-phosphatidylcholine dodecanoyl (DDPC). All formulations listed in Table 1, except #8, contained 10 mM acetate buffer and had a pH of 5.5. Formulations #3 and #6 did not include low molecular weight excipients. Formulation #8 was cell culture media with no EPO-mimetic peptide or low molecular weight excipients and functioned as a negative control.

TABLE 1
Unmodified and PEGylated EPO-mimetic peptide Formulations

| | | M-β-CD | EDTA | DDPC | NaCl |
|---|---|---|---|---|---|
| Compound | Formulation # | (mg/ml) | (mg/ml) | (mg/ml) | (mM) |
| Unmodified EPO- | 1 | 45 | 1 | 1 | 0 |
| mimetic peptide | 2 | 90 | 2 | 2 | 0 |
| ~120 μM or | 3 | 0 | 0 | 0 | 140 |
| 6 mg/ml | | | | | |
| 5 kDa PEGylated | 4 | 45 | 1 | 1 | 0 |
| MC4 RA | 5 | 90 | 2 | 2 | 0 |
| ~120 μM or | 6 | 0 | 0 | 0 | 140 |
| 12 mg/ml | 7 | 0 | 10 | 0 | 0 |
| MatTek Media | 8 | 0 | 0 | 0 | 0 |
| (negative control) | | | | | |

The results of the TEER measurements and permeation assay for formulations shown in Table 1 are summarized below in Table 2. The “Average TEER Measurement” represents the average TEER calculated from measurements taken from experiments performed in triplicate. The greater the TEER value the greater the transcellular resistance.

TABLE 2
TEER Measurements and Permeation Efficacy of Unmodified
and PEGylated EPO-mimetic peptide Formulations

| | Formulation | Average TEER | % |
|---|---|---|---|
| Compound | # | Measurement | Permeation |
| Unmodified EPO-mimetic | 1 | 22.5 | 0% |
| peptide | 2 | 3.6 | 0% |
| ~120 μM or 6 mg/ml | 3 | 286.5 | 0% |
| 5 kDa PEGylated EPO- | 4 | 33.3 | 0.9% |
| mimetic peptide | 5 | 1.2 | 5.2% |
| ~120 μM or 12 mg/ml | 6 | 426.3 | 0% |
| | 7 | 2.1 | 8.9% |
| MatTek Media | 8 | 334.5 | N/A |
| (negative TEER control) | | | |

The results in Table 2 show that the 5 kDa PEGylated EPO-mimetic peptide molecules in enhancer formulations #4, #5 and #7 exhibited greater cellular permeation than the unmodified EPO-mimetic peptide molecules with enhancers (formulations #1 and #2) indicating that conjugation of EPO-mimetic peptide with PEG enhances permeation of EPO-mimetic peptide through an epithelial cell monolayer. Additionally, the 5 kDa PEGylated EPO-mimetic peptide molecules in formulations comprising permeation enhancers (formulations #4 and #5) had a greater cellular permeation than the same molecules without enhancers (formulation #6), indicating that the presence of low molecular weight excipients enhance EPO-mimetic peptide epithelial cell permeation. Optimal cellular permeation was obtained with high concentrations of a chelator (EDTA) (formulation #7).

In comparison, the degree of permeation correlated with the degree of decreased transcellular resistance caused by the formulation. In other words, in general, a low TEER measurement inversely correlated with a high degree of permeation.

These data show that conjugation of EPO-mimetic peptide with PEG enhances its ability to cross the tight junction barrier of an epithelial cell monolayer.

Due to the success of enhancing EPO-mimetic peptide permeation by the covalent addition of 5 kDa PEG to the EPO-mimetic peptide molecule and the presence of the low molecular weight excipient EDTA in the formulation, a second set of formulations containing an increased concentration of EDTA were generated and tested for their ability to decrease TEER and further enhance EPO-mimetic peptide permeation across an epithelial cell monolayer. Table 3 below illustrates both PEGylated and unmodified EPO-mimetic peptide formulations. All EPO-mimetic peptide containing formulations listed in Table 3 were adjusted to pH 5.5. These formulations contained 120 μM of 5 kDa PEGylated or 120 μM unmodified EPO-mimetic peptide with the low molecular weight excipients methyl-β-cyclodextrin (M-β-CD), disodium edentate (EDTA) and L-α-phosphatidylcholine dodecanoyl (DDPC) or the delivery polypeptide PN159 or 120 μM of 5 kDa PEGylated EPO-mimetic peptide without low molecular weight excipients. Formulation #10 was cell culture media with no EPO-mimetic peptide or low molecular weight excipients and functioned as a negative control. Formulation 11 contained only 9% octylphenolpoly(ethyleneglycolether) (TritonX-100™) and functioned as a positive TEER control. Formulations #7, #8 and #9 contained different concentrations of the delivery polypeptide PN159, used herein as a positive control for epithelial cell monolayer permeation, without low molecular weight excipients.

TABLE 3
Unmodified and PEGylated EPO-mimetic peptide Formulations

| | | M-β-CD | EDTA | DDPC | | NaCl |
|---|---|---|---|---|---|---|
| Compound | Formulation # | (mg/ml) | (mg/ml) | (mg/ml) | PN159 | (mM) |
| Unmodified EPO- | 1 | 45 | 10 | 1 | 0 | 0 |
| mimetic peptide | 2 | 90 | 10 | 2 | 0 | 0 |
| ~120 μM or 6 mg/ml | | | | | | |
| 5 kDa PEGylated | 3 | 45 | 10 | 1 | 0 | 0 |
| EPO-mimetic peptide | 4 | 90 | 2 | 2 | 0 | 0 |
| ~120 μM or 12 mg/ml | 5 | 90 | 10 | 2 | 0 | 0 |
| | 6 | 0 | 0 | 0 | 0 | 140 |
| | 7 | 0 | 0 | 0 | 25 μM | 0 |
| | 8 | 0 | 0 | 0 | 50 μM | 0 |
| | 9 | 0 | 0 | 0 | 100 μM | 0 |
| MatTek Media | 10 | 0 | 0 | 0 | 0 | 0 |
| (negative TEER | | | | | | |
| control) | | | | | | |
| 9% Triton X-100 ™ | 11 | 0 | 0 | 0 | 0 | 0 |
| (positive TEER | | | | | | |
| control) | | | | | | |

The results of the TEER measurements and permeation assay for formulations shown in Table 3 are summarized below in Table 4. The “Average TEER Measurement” represents the average TEER calculated from measurements taken from experiments performed in triplicate. The greater the TEER value the greater the transcellular resistance.

TABLE 4
TEER Measurements and Permeation Efficacy of Unmodified
and PEGylated EPO-mimetic peptide Formulations

| | Formulation | Average TEER | |
|---|---|---|---|
| Compound | # | Measurement | % Permeation |
| Unmodified EPO- | 1 | 4.5 | 0% |
| mimetic peptide | 2 | 5.7 | 0% |
| ~120 μM or 6 mg/ml | | | |
| 5 kDa PEGylated EPO- | 3 | 2.1 | 1.0% |
| mimetic peptide | 4 | 6.9 | 3.6% |
| ~120 μAM or 12 mg/ml | 5 | 2.1 | 0.1% |
| | 6 | 95.7 | 0.1% |
| | 7 | 77.7 | 0.2% |
| | 8 | 82.2 | 0.7% |
| | 9 | 414 | 0.3% |
| MatTek Media | 10 | 456.6 | N/A |
| (negative TEER control) | | | |
| 9% Triton X-100 ™ | 11 | 1.5 | N/A |
| (positive TEER control) | | | |

The results in Table 4 show that the 5 kDa PEGylated EPO-mimetic peptide molecules in formulations #3 and #5 comprising permeation enhancers showed greater cellular permeation than the unmodified EPO-mimetic peptide molecules in the same formulations (formulations #1 and #2), which corroborates the results from Table 2 indicating that conjugation of EPO-mimetic peptide with PEG enhances EPO-mimetic peptide permeation through an epithelial cell monolayer. The 5 kDa PEGylated EPO-mimetic peptide formulations #7, #8 and #9 that contain the delivery peptide PN159 showed minimal permeation enhancement. Optimal cellular permeation was obtained with high concentrations of a solubilizer (M-β-CD) as shown by formulation #4. In comparison to 2 mg/ml EDTA in EPO-mimetic peptide formulations, EDTA at or around 10 mg/ml within a EPO-mimetic peptide formulation and in combination with other low molecular weight excipients does not further enhance permeation.

As expected, the MatTek media negative control gave a high TEER value indicating a high degree of transcellular resistance, while the 9% Triton X-100™ showed a low TEER value indicating little to no transcellular resistance.

In comparison, the degree of permeation correlated with the degree of decreased transcellular resistance caused by the formulation. In other words, in general, a low TEER measurement inversely correlated with a high degree of permeation.

These data show further support that conjugation of EPO-mimetic peptide with PEG enhances its ability to cross the tight junction barrier of an epithelial cell monolayer.

### Example 3

Permeation Kinetics of Low and High Molecular Weight PEGylated Forms of an EPO-Mimetic Peptide in the Presence and Absence of Low-Molecular-Weight Excipients

The present example demonstrates that conjugating a low molecular weight PEG to EPO-mimetic peptide significantly enhances the permeation of EPO-mimetic peptide across and epithelial cell monolayer. The instant example evaluated the permeation kinetics of PEGylated EPO-mimetic peptide conjugates having a PEG molecular weight of 2 kDA, 5 kDa, 10 kDa, 20 kDa and 40 kDa in the presence or absence of the low molecular weight excipients methyl-β-cyclodextrin (M-β-CD), disodium edentate (EDTA) and L-α-phosphatidylcholine dodecanoyl (DDPC). Each PEGylated EPO-mimetic peptide form was tested at 12 mg/ml. Table 5 below illustrates the PEGylated EPO-mimetic peptide formulations assayed for TEER. The formulations in Table 5 were not subject to a permeation assay. All EPO-mimetic peptide containing formulations listed in Table 5 were adjusted to pH 5.5. Formulations #11 through #15 did not include any low molecular weight excipients. Formulation #16 was cell culture media with no EPO-mimetic peptide or low molecular weight excipients and functioned as a negative control. Formulation #17 contained only 9% octylphenolpoly(ethyleneglycolether) (TritonX-100™) and functioned as a positive TEER control.

TABLE 5
Low and High Molecular Weight PEG-EPO-mimetic peptide Formulations

| | | | | | Acetate | |
|---|---|---|---|---|---|---|
| Compound | | M-β-CD | EDTA | DDPC | Buffer | NaCl |
| (12 mg/ml) | Formulation # | (mg/ml) | (mg/ml) | (mg/ml) | (mM) | (mM) |
| 2 kDa PEG-EPO-mimetic | 1 | 90 | 10 | 2 | 10 | 0 |
| peptide | | | | | | |
| 5 kDa PEG-EPO-mimetic | 2 | 90 | 10 | 2 | 10 | 0 |
| peptide | | | | | | |
| 10 kDa PEG-EPO-mimetic | 3 | 90 | 10 | 2 | 10 | 0 |
| peptide | | | | | | |
| 20 kDa PEG-EPO-mimetic | 4 | 90 | 10 | 2 | 10 | 0 |
| peptide | | | | | | |
| 40 kDa PEG-EPO-mimetic | 5 | 90 | 10 | 2 | 10 | 0 |
| peptide | | | | | | |
| 2 kDa PEG-EPO-mimetic | 6 | 0 | 10 | 0 | 0 | 0 |
| peptide | | | | | | |
| 5 kDa PEG-EPO-mimetic | 7 | 0 | 10 | 0 | 0 | 0 |
| peptide | | | | | | |
| 10 kDa PEG-EPO-mimetic | 8 | 0 | 10 | 0 | 0 | 0 |
| peptide | | | | | | |
| 20 kDa PEG-EPO-mimetic | 9 | 0 | 10 | 0 | 0 | 0 |
| peptide | | | | | | |
| 40 kDa PEG-EPO-mimetic | 10 | 0 | 10 | 0 | 0 | 0 |
| peptide | | | | | | |
| 2 kDa PEG-EPO-mimetic | 11 | 0 | 0 | 0 | 0 | 140 |
| peptide | | | | | | |
| 5 kDa PEG-EPO-mimetic | 12 | 0 | 0 | 0 | 0 | 140 |
| peptide | | | | | | |
| 10 kDa PEG-EPO-mimetic | 13 | 0 | 0 | 0 | 0 | 140 |
| peptide | | | | | | |
| 20 kDa PEG-EPO-mimetic | 14 | 0 | 0 | 0 | 0 | 140 |
| peptide | | | | | | |
| 40 kDa PEG-EPO-mimetic | 15 | 0 | 0 | 0 | 0 | 140 |
| peptide | | | | | | |
| MatTek Media | 16 | 0 | 0 | 0 | 0 | 0 |
| (negative TEER control) | | | | | | |
| 9% Triton X-100 ™ | 17 | 0 | 0 | 0 | 0 | 0 |
| (positive TEER control) | | | | | | |

The results of the TEER measurements for formulations shown in Table 5 are summarized below in Table 6. The “Average TEER Measurement” represents the average TEER calculated from measurements taken from experiments performed in triplicate. The greater the TEER value the greater the transcellular resistance.

TABLE 6
TEER Measurements of Low and High Molecular
Weight PEGylated EPO-mimetic peptide Formulations

| | | Average |
|---|---|---|
| Compound | Formulation # | TEER Measurement |
| 2 kDa PEG-EPO-mimetic peptide | 1 | 1.6 |
| 5 kDa PEG-EPO-mimetic peptide | 2 | 1.8 |
| 10 kDa PEG-EPO-mimetic peptide | 3 | 2 |
| 20 kDa PEG-EPO-mimetic peptide | 4 | 1.6 |
| 40 kDa PEG-EPO-mimetic peptide | 5 | 2 |
| 2 kDa PEG-EPO-mimetic peptide | 6 | 2.2 |
| 5 kDa PEG-EPO-mimetic peptide | 7 | 2.8 |
| 10 kDa PEG-EPO-mimetic peptide | 8 | 2.4 |
| 20 kDa PEG-EPO-mimetic peptide | 9 | 1.8 |
| 40 kDa PEG-EPO-mimetic peptide | 10 | 2.2 |
| 2 kDa PEG-EPO-mimetic peptide | 11 | 621.4 |
| 5 kDa PEG-EPO-mimetic peptide | 12 | 402 |
| 10 kDa PEG-EPO-mimetic peptide | 13 | 518.6 |
| 20 kDa PEG-EPO-mimetic peptide | 14 | 558.2 |
| 40 kDa PEG-EPO-mimetic peptide | 15 | 537 |
| MatTek Media | 16 | 543 |
| (negative TEER control) | | |
| 9% Triton X-100 ™ | 17 | 1.2 |
| (positive TEER control) | | |

The results in Table 6 show that low molecular weight excipients in EPO-mimetic peptide formulations (formulations #1 through #10) significantly reduce transcellular electrical resistance compared to EPO-mimetic peptide formulations without low molecular weight excipients (formulations #11 through #15). Additionally, formulations containing only the low molecular weight excipient EDTA (formulations #6 through #10) worked as effectively as those formulations containing all three low molecular weight excipients (i.e., M-β-CD, DDPC and EDTA; formulations #1 through #5) to decrease resistance.

Based on the foregoing results, only the low and high molecular weight PEGylated EPO-mimetic peptide formulations containing 10 mg/ml EDTA were assayed for EPO-mimetic peptide epithelial cell monolayer permeation efficacy. Table 7 below illustrates the EDTA only PEGylated EPO-mimetic peptide formulations. All EPO-mimetic peptide containing formulations listed in Table 7 were adjusted to pH 5.5. Each PEGylated EPO-mimetic peptide form was tested at 12 mg/ml. Formulation #6 was cell culture media with no EPO-mimetic peptide or EDTA and functioned as a negative control. Formulation 7 contained only 9% Octylphenolpoly(ethyleneglycolether)ₓ (TritonX-100™).

TABLE 7
Low and High Molecular Weight PEGylated
EPO-mimetic peptide EDTA Containing Formulations

| Compound | Formulation | EDTA | Acetate |
|---|---|---|---|
| (12 mg/ml) | # | (mg/ml) | Buffer (mM) |
| 2 kDa PEG-EPO-mimetic peptide | 1 | 10 | 10 |
| 5 kDa PEG-EPO-mimetic peptide | 2 | 10 | 10 |
| 10 kDa PEG-EPO-mimetic peptide | 3 | 10 | 10 |
| 20 kDa PEG-EPO-mimetic peptide | 4 | 10 | 10 |
| 40 kDa PEG-EPO-mimetic peptide | 5 | 10 | 10 |
| MatTek Media | 6 | 0 | 0 |
| (negative TEER control) | | | |
| 9% Triton X-100 ™ | 7 | 0 | 0 |
| (positive TEER control) | | | |

The results of the permeation assay for formulations shown in Table 7 are summarized below in Table 8.

TABLE 8
Permeation Efficacy of Low and High Molecular Weight
PEGylated EPO-mimetic peptide EDTA Containing Formulations

| Compound (12 mg/ml) | Formulation # | % Permeation |
|---|---|---|
| 2 kDa PEG-EPO-mimetic peptide | 1 | 20.8% |
| 5 kDa PEG-EPO-mimetic peptide | 2 | 9.8% |
| 10 kDa PEG-EPO-mimetic peptide | 3 | 5.6% |
| 20 kDa PEG-EPO-mimetic peptide | 4 | 0.7% |
| 40 kDa PEG-EPO-mimetic peptide | 5 | 0.1% |

The results shown in Table 8 indicate an inverse relationship between the degree of permeation and the molecular weight of the covalently linked PEG moiety on the EPO-mimetic peptide molecule. As the molecular weight of the PEG moiety increases, the level of permeation decreases. The low molecular weight 2 kDa PEGylated form of EPO-mimetic peptide has the greatest degree of permeation at approximately 21%. The optimal cellular permeation was obtained with a chelator (e.g., EDTA).

Thus, these data show the surprising and unexpected discovery that conjugation of a low molecular weight PEG to a EPO-mimetic peptide, for example a 2 kDa PEG, in combination with a chelator significantly enhances the EPO-mimetic peptide molecule's ability to permeate an epithelial cell monolayer.

### Example 4

### TEER Measurements of Low and High Molecular Weight PEGylated Forms of a MC4-RA in the Presence or Absence of Low Molecular Weight Excipients

The present example demonstrates that increased concentrations of high molecular weight PEGylated forms of EPO-mimetic peptide do not alter TEER value compared to lower concentrations of the same molecular weight PEGylated EPO-mimetic peptide form. The instant example evaluated the permeation kinetics of PEGylated EPO-mimetic peptide conjugates having a PEG molecular weight of 2 kDa, 5 kDa, 10 kDa, 20 kDa and 40 kDa in the presence or absence of the low molecular weight excipients M-β-CD, disodium EDTA and DDPC. The instant Example differs from the prior Example in that both the 20 kDa and 40 kDa PEGylated forms of EPO-mimetic peptide in the instant Example were assayed for TEER at a higher concentration (24 mg/ml). The 2 kDa, 5 kDa and 10 kDa PEGylated EPO-mimetic peptide molecules were again assayed for TEER at 12 mg/ml. Table 9 below illustrates the PEGylated EPO-mimetic peptide formulations assayed for TEER. All EPO-mimetic peptide containing formulations listed in Table 9 were adjusted to pH 5.5. Formulations #11 through #1 5 did not include any low molecular weight excipients. Formulation #18 was cell culture media with no EPO-mimetic peptide or low molecular weight excipients and functioned as a negative control. Formulation #19 contained only 9% octylphenolpoly(ethyleneglycolether) (TritonX-100™) and functioned as a positive TEER control.

TABLE 9
Low and High Molecular Weight EPO-mimetic peptide Formulations

| Compound | | M-β-CD | | | Acetate Buffer | |
|---|---|---|---|---|---|---|
| (concentration) | Formulation # | (mg/ml) | EDTA (mg/ml) | DDPC (mg/ml) | (mM) | NaCl (mM) |
| 2 kDa PEG-EPO-mimetic peptide | 1 | 90 | 10 | 2 | 10 | 0 |
| (12 mg/ml) | | | | | | |
| 5 kDa PEG-EPO-mimetic peptide | 2 | 90 | 10 | 2 | 10 | 0 |
| (12 mg/ml) | | | | | | |
| 10 kDa PEG-EPO-mimetic peptide | 3 | 90 | 10 | 2 | 10 | 0 |
| (12 mg/ml) | | | | | | |
| 20 kDa PEG-EPO-mimetic peptide | 4 | 90 | 10 | 2 | 10 | 0 |
| (24 mg/ml) | | | | | | |
| 40 kDa PEG-EPO-mimetic peptide | 5 | 90 | 10 | 2 | 10 | 0 |
| (24 mg/ml) | | | | | | |
| 2 kDa PEG-EPO-mimetic peptide | 6 | 0 | 10 | 0 | 0 | 0 |
| (24 mg/ml) | | | | | | |
| 5 kDa PEG-EPO-mimetic peptide | 7 | 0 | 10 | 0 | 0 | 0 |
| (12 mg/ml) | | | | | | |
| 10 kDa PEG-EPO-mimetic peptide | 8 | 0 | 10 | 0 | 0 | 0 |
| (12 mg/ml) | | | | | | |
| 20 kDa PEG-EPO-mimetic peptide | 9 | 0 | 10 | 0 | 0 | 0 |
| (24 mg/ml) | | | | | | |
| 40 kDa PEG-EPO-mimetic peptide | 10 | 0 | 10 | 0 | 0 | 0 |
| (24 mg/ml) | | | | | | |
| 2 kDa PEG-EPO-mimetic peptide | 11 | 0 | 0 | 0 | 0 | 140 |
| (12 mg/ml) | | | | | | |
| 5 kDa PEG-EPO-mimetic peptide | 12 | 0 | 0 | 0 | 0 | 140 |
| (12 mg/ml) | | | | | | |
| 10 kDa PEG-EPO-mimetic peptide | 13 | 0 | 0 | 0 | 0 | 140 |
| (12 mg/ml) | | | | | | |
| 20 kDa PEG-EPO-mimetic peptide | 14 | 0 | 0 | 0 | 0 | 140 |
| (24 mg/ml) | | | | | | |
| 40 kDa PEG-EPO-mimetic peptide | 15 | 0 | 0 | 0 | 0 | 140 |
| (24 mg/ml) | | | | | | |
| 2 kDa PEG-EPO-mimetic peptide | 16 | 45 | 1 | 1 | 10 | 0 |
| (12 mg/ml) | | | | | | |
| 5 kDa PEG-EPO-mimetic peptide | 17 | 45 | 1 | 1 | 10 | 0 |
| (12 mg/ml) | | | | | | |
| MatTek Media | 18 | 0 | 0 | 0 | 0 | 0 |
| (negative TEER control) | | | | | | |
| 9% Triton X-100 ™ | 19 | 0 | 0 | 0 | 0 | 0 |
| (positive TEER control) | | | | | | |

The results of the TEER measurements for formulations shown in Table 9 are summarized below in Table 10. The “Average TEER Measurement” represents the average TEER calculated from measurements taken from experiments performed in triplicate. The greater the TEER value the greater the transcellular resistance.

TABLE 10
TEER Measurements of Low and High Molecular
Weight PEGylated EPO-mimetic peptide Formulations

| | | Average |
|---|---|---|
| Compound (concentration) | Formulation # | TEER Measurement |
| 2 kDa PEG-EPO-mimetic peptide | 1 | 1.6 |
| (12 mg/ml) | | |
| 5 kDa PEG-EPO-mimetic peptide | 2 | 1.8 |
| (12 mg/ml) | | |
| 10 kDa PEG-EPO-mimetic peptide | 3 | 2 |
| (12 mg/ml) | | |
| 20 kDa PEG-EPO-mimetic peptide | 4 | 2 |
| (24 mg/ml) | | |
| 40 kDa PEG-EPO-mimetic peptide | 5 | 1.6 |
| (24 mg/ml) | | |
| 2 kDa PEG-EPO-mimetic peptide | 6 | 2.2 |
| (12 mg/ml) | | |
| 5 kDa PEG-EPO-mimetic peptide | 7 | 2.8 |
| (12 mg/ml) | | |
| 10 kDa PEG-EPO-mimetic peptide | 8 | 2.4 |
| (12 mg/ml) | | |
| 20 kDa PEG-EPO-mimetic peptide | 9 | 2.2 |
| (24 mg/ml) | | |
| 40 kDa PEG-EPO-mimetic peptide | 10 | 1.8 |
| (24 mg/ml) | | |
| 2 kDa PEG-EPO-mimetic peptide | 11 | 621.4 |
| (12 mg/ml) | | |
| 5 kDa PEG-EPO-mimetic peptide | 12 | 402 |
| (12 mg/ml) | | |
| 10 kDa PEG-EPO-mimetic peptide | 13 | 518.6 |
| (12 mg/ml) | | |
| 20 kDa PEG-EPO-mimetic peptide | 14 | 537 |
| (24 mg/ml) | | |
| 40 kDa PEG-EPO-mimetic peptide | 15 | 558.2 |
| (24 mg/ml) | | |
| 2 kDa PEG-EPO-mimetic peptide | 16 | 543 |
| (12 mg/ml) | | |
| 5 kDa PEG-EPO-mimetic peptide | 17 | 1.2 |
| (12 mg/ml) | | |

The results in Table 10 show that PEGylation combined with low molecular weight excipients greatly decreased transcellular resistance. In particular, a chelator (e.g., EDTA) alone, or in combination with other low molecular weight excipients, for example M-β-CD and DDPC, and acetate, pH 5.5 was sufficient to induce a loss of resistance across the cellular layer. Further, TEER values were unaffected with increased concentration of the high molecular weight PEGylated forms of EPO-mimetic peptide. These data confirm the results shown in previous Example sections of the instant application.

### Example 5

### In Vitro Potency and Melanocortin Receptor Agonist (MC4-RA) Specificity of a Low Molecular Weight PEGylated MC4-RA and an Unmodified MC4-RA

The present example demonstrates that a low molecular weight PEGylated MC4-RA exhibits greater selectivity than the unmodified MC4-RA in stimulating members of the melanocortin cell surface receptor family. An ideal property of any therapeutic agent is target specificity as induction, for example, of unwanted cell surface receptors and/or cell signaling pathways may lead to deleterious outcomes in the patient subject. In this case, MC4-RA is used as a therapeutic agent to specifically target the melanocortin-4 cell surface receptor. The instant example employs a cAMP assay to compare both the melanocortin-4 receptor stimulating potency and melanocortin receptor specificity of a 2 kDa PEGylated MC4-RA (low molecular weight form) and a unmodified MC4-RA in HEK293 cells.

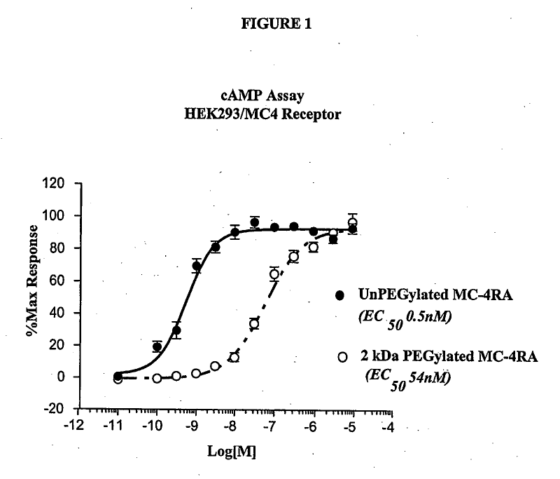

Potency was measured as the ability of the 2 kDa PEGylated MC4-RA or the unmodified MC4-RA to stimulate cAMP production in cells expressing the melanocortin-4 cell receptor (MC4 receptor). The 2 kDa PEGylated and unmodified MC4-RA were incubated in a concentration range of approximately 1×10⁻¹¹ to 1×10⁻⁵ M with HEK293 cells expressing the MC4 receptor. The experiment was performed in triplicate and cAMP levels were measured with the cAMP Tropix assay kit. The results are shown in FIG. 1. The maximum quantity of cAMP was normalized to 100% or “% Max Response” and the concentration of 2 kDa PEGylated or unmodified MC4-RA is shown as the log of the Molar concentration. As shown in FIG. 1, the effective concentration to reach a 50% response level (EC₅₀) for the low molecular weight form of MC4-RA (EC₅₀=54 nM) was higher than the unmodified MC4-RA (EC₅₀=0.5 nM) indicating that the 2 kDa PEGylated MC4-RA is a less potent activator of the MC-4 receptor compared to the unmodified MC4-RA in an in vitro assay system. However, in vivo results show that the differential therapeutic efficacy between the 2 kDa PEGylated MC4-RA and the unmodified MC4-RA is minimal (refer to Example 6) indicating that the discrepancy in MC4 receptor stimulating activity between the PEGylated MC4-RA molecule and the unmodified MC4-RA molecule observed in vitro does represent a limitation on the therapeutic activity of the low molecular weight PEGylated MC4-RA molecule.

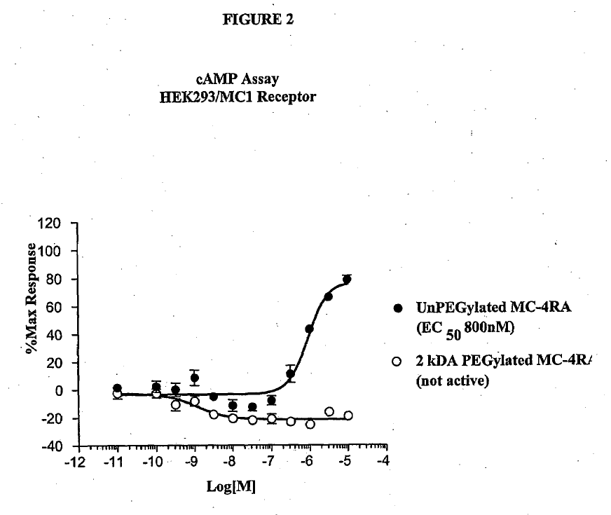

The degree of MC4 receptor specificity of the 2 kDa PEGylated MC4-RA and the unmodified MC4-RA was compared. Again, the ability to stimulate cAMP production in cells in vitro was assayed; however, the HEK293 cells were not expressing the MC4 receptor but the related cell surface receptor family member, melanocortin-1 (MC1 receptor). In this instance, a measured increase in cAMP levels would indicate a lack of MC4 receptor specificity. The 2 kDa PEGylated MC4-RA and the unmodified MC4-RA were incubated in a concentration range of approximately 1×10⁻¹¹ to 1×10⁻⁵ M with HEK293 cells expressing the MC1 receptor. The experiment was performed in triplicate and cAMP levels were measured with the cAMP Tropix assay kit. The results are shown in FIG. 2. The maximum quantity of cAMP was normalized to 100% or “% Max Response” and the concentration of 2 kDa PEGylated or unmodified MC4-RA is shown as the log of the Molar concentration. As shown if FIG. 2, the effective concentration to reach a 50% response level (EC₅₀) for the unmodified MC4-RA was approximately 800 nM while the low molecular weight PEGylated MC4-RA did not induce cAMP levels at the concentrations tested indicating the PEGylation significantly enhanced the specificity of MC4-RA for the MC4 receptor.

### Example 6

### Mice Administered a Low Molecular Weight PEGylated MC4-RA had Reduced Cumulative Food Intake

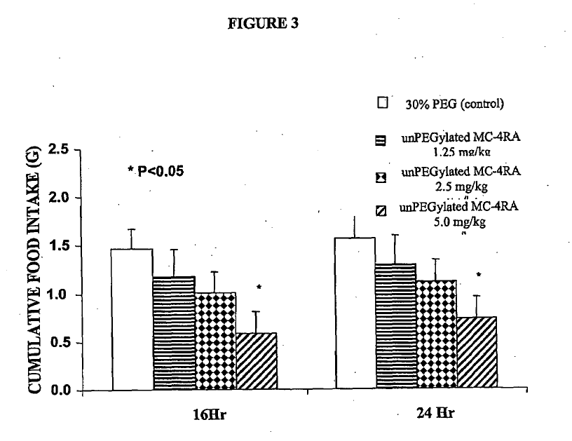

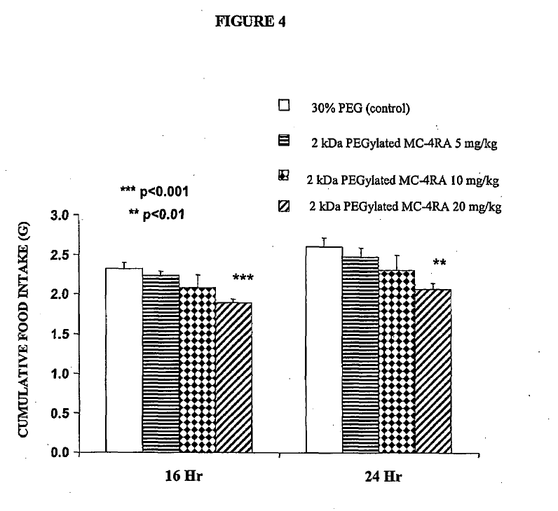

The present example demonstrates that the low molecular weight PEGylated MC4-RA molecules when administered to a mammalian subject significantly reduced cumulative food intake of that subject 16 and 24 hours after dose administration. The effect of the low molecular weight PEGylated MC4-RA and unmodified MC4-RA on food intake was evaluated under regular light cycle in male DOI mice (obesity mouse model system). Control mice were administered a 30% PEG formulation. For the unmodified MC4-RA in vivo study, individual subjects categorized into three separate study groups, based on dosage levels, were administered 1.25 mg/kg, 2.5 mg/kg or 5 mg/kg of unmodified MC4-RA. For the 2 kDa PEGylated MC4-RA in vivo study, individual subjects categorized into three separate study groups, again based on dosage levels, were administered 5 mg/kg, 10 mg/kg and 20 mg/kg 2 kDa PEGylated MC4-RA. The effective does is higher for the PEGylated MC4-RA due to the tripled molecular weight resulting from the conjugation of a 2 kDa PEG to the MC4-RA molecule. The results are shown in FIG. 3 and FIG. 4. The high dose group for both the 2 kDa PEGylated MC4-RA and the unmodified MC4-RA showed significant reduction on cumulative food intake 16 and 24 hours after dose administration.

These data show that a low molecular weight PEGylated MC4-RA molecule when administered to a mammalian subject significantly reduces cumulative food intake.

1-49. (canceled)

50. A biological agent for intranasal mucosal delivery, comprising peptide YY₃₋₃₆ conjugated to at least one poly(alkylene oxide) chain having a molecular size less than about 20 kiloDaltons.

51. The biological agent of claim 50, wherein the poly(alkylene oxide) chain is a polyethylene glycol chain having a molecular size from about 0.2 to less than about 5 kiloDaltons.

52. The biological agent of claim 50, wherein the poly(alkylene oxide) chain is a polyethylene glycol chain having a molecular size of 0.2 kiloDaltons, and wherein the polyethylene glycol chain enhances intranasal permeation of the peptide YY₃₋₃₆.

53. The biological agent of claim 50, wherein the poly(alkylene oxide) chain is a polyethylene glycol chain having a polydispersity value (Mw/Mn) of less than 1.20.

54. A pharmaceutical formulation comprising the biological agent of claim 50, and an enhancer of permeation.

55. The pharmaceutical formulation of claim 54, wherein the enhancer of permeation is selected from the group consisting of surface acting agents, chelating agents, solubilizing agents, one or more polyol, and combinations thereof.

56. The pharmaceutical formulation of claim 55, wherein the surface acting agent is selected from the group consisting of a nonionic polyoxyethylene ether, bile salts such as sodium glycocholate (SGC), deoxycholate (DOC), derivatives of fusidic acid, or sodium taurodihydrofusidate (STDHF), L-α-phosphatidylcholine dodecanoyl (DDPC), polysorbate 80 and polysorbate 20, cetyl alcohol, polyvinylpyrolidone (PVP), polyvinyl alcohol (PVA), lanolin alcohol, and sorbitan monooleate.

57. The pharmaceutical formulation of claim 55, wherein the chelating agent is EDTA.

58. The pharmaceutical formulation of claim 55, wherein the solubilizing agent is selected from the group consisting of cyclodextran, hydroxypropyl-β-cyclodextran, sulfobutylether-β-cyclodextran and methyl-β-cyclodextrin, most preferably methyl-β-cyclodextrin.

59. A method for treating a peptide YY₃₋₃₆ disease in a mammalian subject comprising administering an effective amount of a formulation according to claim 54 to a subject in need thereof, wherein the biological agent is present at a concentration greater than about 2.5 mM.

60. The method of claim 59, wherein the disease is obesity and/or reducing food intake.

61. A biological agent for intranasal mucosal delivery, comprising parathyroid hormone₁₋₃₄ conjugated to a at least one poly(alkylene oxide) chain having a molecular size less than about 20 kiloDaltons.

62. The biological agent of claim 61, wherein the poly(alkylene oxide) chain is a polyethylene glycol chain having a molecular size from about 0.2 to less than about 5 kiloDaltons.

63. The biological agent of claim 61, wherein the poly(alkylene oxide) chain is a polyethylene glycol chain having a molecular size of 2 kDa, and wherein the polyethylene glycol chain enhances intranasal permeation of PTH₁₋₃₄.

64. The biological agent of claim 61, wherein the poly(alkylene oxide) chain is a polyethylene glycol chain having a molecular size of 1.8 kDa, and wherein the polyethylene glycol chain enhances intranasal permeation of PTH₁₋₃₄.

65. The biological agent of claim 61, wherein the poly(alkylene oxide) chain is a polyethylene glycol chain having a polydispersity value (Mw/Mn) of less than 1.20.

66. A pharmaceutical formulation comprising the biological agent of claim 61, and an enhancer of permeation.

67. The pharmaceutical formulation of claim 66, wherein the enhancer of permeation is selected from the group consisting of surface acting agents, chelating agents, solubilizing agents, one or more polyol, and combinations thereof.

68. The pharmaceutical formulation of claim 67, wherein the surface acting agent is selected from the group consisting of a nonionic polyoxyethylene ether, bile salts such as sodium glycocholate (SGC), deoxycholate (DOC), derivatives of fusidic acid, or sodium taurodihydrofusidate (STDHF), L-α-phosphatidylcholine dodecanoyl (DDPC), polysorbate 80 and polysorbate 20, cetyl alcohol, polyvinylpyrolidone (PVP), polyvinyl alcohol (PVA), lanolin alcohol, and sorbitan monooleate.

69. The pharmaceutical formulation of claim 67, wherein the chelating agent is EDTA.

70. The pharmaceutical formulation of claim 67, wherein the solubilizing agent is selected from the group consisting of cyclodextran, hydroxypropyl-β-cyclodextran, sulfobutylether-β-cyclodextran and methyl-β-cyclodextrin, most preferably methyl-β-cyclodextrin.

71. A method for treating a parathyroid hormone₁₋₃₄ related disease in a mammalian subject comprising administering an effective amount of a formulation according to claim 66 to a subject in need thereof, wherein the biological agent is present at a concentration greater than about 2.5 mM.
